(19) United States
(12) Patent Application Publication
(10) Pub. No.: US 2011/0130840 A1
(43) Pub. Date: Jun. 2, 2011

(54) SOFT TISSUE ATTACHMENT DEVICE AND METHOD

(76) Inventor: Shervin V. Oskouei, Atlanta, GA (US)

(21) Appl. No.: 12/784,081

(22) Filed: May 20, 2010

Related U.S. Application Data
(60) Provisional application No. 61/179,961, filed on May 20, 2009.

(51) Int. Cl. A61F 2/30

(52) U.S. Cl. 623/18.11

(57) ABSTRACT

An attachment device that secures soft tissue to an orthopedic prosthetic implant or bone. The attachment device includes a receptor surface, an attachment plate with an attachment surface, and fasteners. The receptor surface can be formed on the implant or on a separate receptor plate. The receptor surface accepts the soft tissue and/or a bony structure, the attachment plate at least partially covers the receptor surface, and the fasteners secure them together to compress and hold in place the soft tissue and/or a bony structure.

### TECHNICAL FIELD

The present invention relates generally to orthopedic surgical devices and procedures and, in particular, to attachments for connecting soft tissue to orthopedic prosthetic implants.

### BACKGROUND

In orthopedic surgery, it is common to attach soft tissue to prosthetic implants. For example, during surgery to replace a knee joint with a prosthetic joint, the patellar and hamstring tendons are removed from the tibia, the end-parts of the bones are removed, the prosthetic knee joint is implanted, and the tendons are reattached to the prosthetic joint. However, there are currently no standard practices for reattaching tendons to prosthetic joints. Many implants include hooks or holes at tendon reattachment sites, and it is left to the surgeon to devise a suitable way to use them to reattach the tendons. For example, many surgeons will route the tendons around or through the reattachment hooks or holes and secure them in place with sutures. The known ways of reattaching the tendons have not proven as effective or satisfactory as would be desired.

Accordingly, it can be seen that needs exist for improved ways to attach soft tissue during orthopedic surgery. It is to the provision of solutions meeting this and other needs that the present invention is primarily directed.

### SUMMARY

Generally described, the present invention relates to an attachment device that secures soft tissue (with or without a bony structure) to an orthopedic prosthetic implant or bone. The attachment device includes a receptor surface, an attachment plate with an attachment surface, and fasteners. The receptor surface can be formed on the implant or on a separate receptor plate. The receptor surface accepts the soft tissue and/or a bony structure, the attachment plate at least partially covers the receptor surface, and the fasteners secure them together to compress and hold in place the soft tissue and/or a bony structure.

The specific techniques and structures employed by the invention to improve over the drawbacks of the prior devices and accomplish the advantages described herein will become apparent from the following detailed description of the example embodiments of the invention and the appended drawings and claims.

### BRIEF DESCRIPTION OF THE DRAWINGS

FIG. 1 is a side view of an orthopedic prosthetic implant with an attachment device according to a first example embodiment of the present invention, showing the attachment device securing soft tissue to the implant.

FIG. 2 is a front view of the orthopedic prosthetic implant and a receptor surface of the attachment device of FIG. 1.

FIG. 3 is a perspective view of the orthopedic prosthetic implant and attachment device of FIG. 1.

FIG. 4 is a side view of the attachment device of FIG. 1, shown compressing soft tissue and a bony structure.

FIG. 5 is a side view of the attachment device of FIG. 1, shown compressing only a bony structure to which soft tissue is attached.

FIG. 6 is a perspective view of an orthopedic prosthetic implant with an attachment device according to a second example embodiment of the present invention.

FIG. 7 is a side view of an orthopedic prosthetic implant with an attachment device according to a third example embodiment of the present invention.

FIG. 8 is a front view of the orthopedic prosthetic implant and a receptor surface of the attachment device of FIG. 7.

FIG. 9 is a side view of an orthopedic prosthetic implant with an attachment device according to a fourth example embodiment of the present invention.

FIG. 10 is a front view of the orthopedic prosthetic implant and a receptor surface of the attachment device of FIG. 9.

FIG. 11 is a side view of an attachment device according to a fifth example embodiment of the present invention.

FIG. 12 is a side view of a modified orthopedic prosthetic implant and attachment device according to a first alternative embodiment of the present invention.

FIG. 13 is a perspective view of the orthopedic prosthetic implant and a receptor surface of the attachment device of FIG. 12.

FIG. 14 is a side view of a modified orthopedic prosthetic implant and attachment device according to a second alternative embodiment of the present invention.

FIG. 15 is a perspective view of the orthopedic prosthetic implant and a receptor surface of the attachment device of FIG. 14.

FIG. 16 is a side view of a modified orthopedic prosthetic implant and attachment device according to a third alternative embodiment of the present invention.

FIG. 17 is a side view of a modified orthopedic prosthetic implant and attachment device according to a fourth alternative embodiment of the present invention.

FIG. 18 is a side view of a modified orthopedic prosthetic implant and attachment device according to a fifth alternative embodiment of the present invention.

FIG. 19 is a perspective view of a modified orthopedic prosthetic implant and attachment device according to a sixth alternative embodiment of the present invention.

FIG. 20 is a perspective view of a modified orthopedic prosthetic implant and attachment device according to a seventh alternative embodiment of the present invention.

FIG. 21 is a perspective view of a modified orthopedic prosthetic implant and attachment device according to an eighth alternative embodiment of the present invention.

### DETAILED DESCRIPTION OF EXAMPLE EMBODIMENTS

Generally described, the present invention relates to devices and methods for attaching soft tissue (with or without a bony structure) during orthopedic surgery. In various example embodiments described herein, the devices and methods are designed for attaching soft tissue to prosthetic implants. In other example embodiments described herein, the devices and methods are designed for attaching soft tissue (with or without a bony structure) to bone or prosthetic implants. In the depicted embodiments, the soft tissue being attached is tendons. As used herein, the term “soft tissue” is meant to also include capsules, musculotendinous units, muscles, ligaments, or other soft tissue in the body of humans or other animals, with or without bony structures. Bony structures (also referred to as bony attachments) are included, for example, when the surgeon slices a sliver of bone underlying where a tendon attaches to the bone when detaching the tendon from the bone before reconstructing the joint, thereby maintaining the tendon-to-bone attachment.

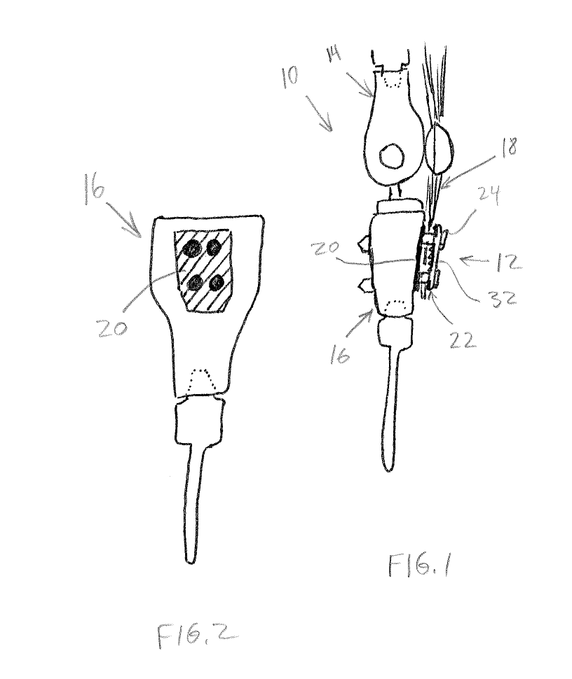

Referring now to the drawing figures, FIGS. 1-5 show an orthopedic prosthetic implant 10 with an attachment device 12 according to a first example embodiment of the present invention. In this embodiment, the implant 10 is a knee-joint arthroplasty implant used to reconstruct a knee. The implant 10 includes a proximal tibial component 14 and a distal femur component 16. The soft tissue 18 reattached is the patellar tendon, with or without a portion of the tibial tubercle bony structure.

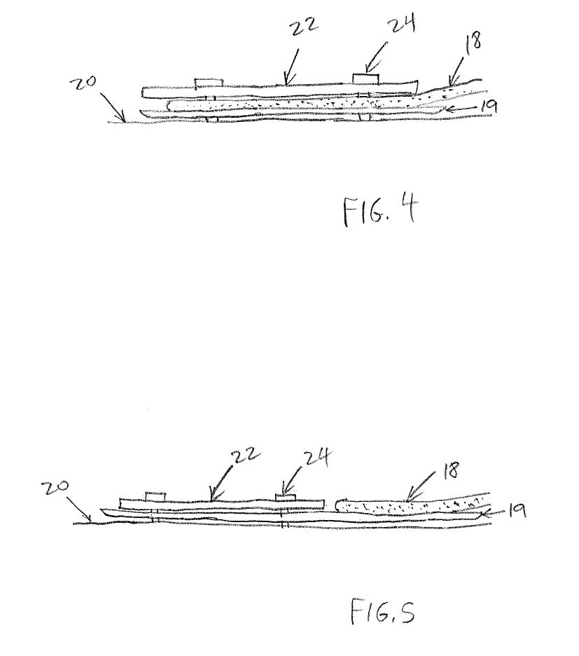

The attachment device 12 includes a receptor surface 20, an attachment plate 22, and fasteners 24. The receptor surface 20 accepts the soft tissue 18. The attachment plate 22 mounts over the receptor surface 20 to sandwich the soft tissue 18 between them. And the fasteners 24 secure the attachment plate 22 to the receptor surface 20. The attachment plate 22 and the receptor surface 20 can be used to sandwich the soft tissue 18, with or without a bony structure, or to sandwich only a bony structure to which soft tissue is attached, with the tendon not actually being between them. FIG. 1 shows the attachment plate 22 and the receptor surface 20 sandwiching only the soft tissue 18, FIG. 4 shows them sandwiching the soft tissue and a bony structure 19 (bone and tissue in-growth are more predictable, and with the fasteners extending through the soft tissue and the bony structure, the securement is excellent), and FIG. 5 shows them sandwiching only the bony structure.

In the depicted embodiment, the receptor surface 20 is formed on the proximal tibial component 14 of the implant 10. For example, during manufacture the implant 10 can be machined to form the receptor surface 20, or the implant can be cast or molded with the receptor surface integrally formed on it in one step. The receptor surface 20 can have a clearly identified boundary to facilitate proper placement of the tendon on it. For example, the receptor surface 20 can be inset into the implant 10 with the inset edges clearly identifying its boundary, or the boundary can be marked by etching or by a slightly raised peripheral ridge. In alternative embodiments, the receptor surface is formed at another location on the proximal tibial component from what is shown and/or formed on the distal femur component. And in other alternative embodiments, the receptor surface is formed on a separate receptor plate that is attached to the implant (with or without being inset) or bone.

The attachment plate 22 of the depicted embodiment is a thin panel-like structure. For example, the attachment plate 22 can have a thickness of about several microns to about one centimeter. In alternative embodiments, the attachment plate is provided by a latticed, grid-like, peripheral-frame, or perforated structure. In other alternative embodiments, the attachment plate 22 is provided by a plurality of plates positioned adjacent to each other but not connected to each other, each secured to the receptor surface 20 by at least one of the fasteners 24. The attachment plate 22 can have a greater and/or irregular thickness relative to that shown, though this will increase its profile.

The receptor surface 20 and the attachment plate 22 can be made of the same material that all or part of the implant 10 is made of, or they can be made of different materials such as a metal, hard plastic, or ceramic material. Conventional orthopedic prosthetic implants are made of metals such as titanium and titanium alloys, tantalum, cobalt-chrome, stainless steel, etc. In some embodiments, the receptor surface 20 and/or the attachment plate 22, or at least portions of them that contact the soft tissue 18 (e.g., an attachment surface 26 of the attachment plate), are made of a highly porous material. The material is selected with a porosity high enough to promote (or at least allow) in-growth of the soft tissue 18, thereby improving the biologic fixation of the soft tissue or bone to the receptor surface 20 and/or the attachment plate 22. Suitable highly porous materials that are commercially available include, but are not limited to, TRABECULAR METAL tantalum provided by ZIMMER of Warsaw, Ind.; TRITANIUM titanium provided by STRYKER BIOTECH of Hopkinton, Mass.; and BIOFOAM titanium foam provided by WRIGHT MEDICAL TECHNOLOGY of Arlington, Tenn. Additionally or alternatively, the receptor surface 20 and/or the attachment surface 26 can be coated with one ore more agents to induce soft tissue and/or bone in-growth, such as hydroxyapatite (HA).

The fasteners 24 secure together the receptor surface 20 and the attachment plate 22. In the depicted embodiment, the fasteners 24 are provided by screws that are received through holes 28 in the attachment plate 22 and threaded holes 30 in the receptor surface 20. The receptor holes 30 and the fasteners 24 can be designed and selected so that fasteners extend all the way through the implant (e.g., with through-holes and longer screws), as shown in FIG. 1, or so that the fasteners extend only partially through the implant such that they do not extend out the opposite side (e.g., with recess-holes and shorter screws). In addition, the attachment and receptor holes 28 and 30 can be positioned within the peripheral boundaries of the attachment and receptor surfaces 26 and 20 so that the fasteners 24 extend through the soft tissue 18 to help secure it in place, as depicted. Furthermore, the fastener screws 24 can each include one or more elastic members, such as soft plastic (e.g., polyethylene) or rubber lateral inserts, that absorb impact vibrations and forces so that the fastener screws do not easily back out and loosen the securement. The implant 10 and attachment device 12 can be designed with four attachment holes 28, receptor holes 30, and fasteners 24, as shown, or with more or fewer of these. For example, the implant 10 and attachment device 12 can be designed with only two, or even only one, of each of these, though including at least two of these tends to provide good results. In alternative embodiments, the fasteners are provided by snap-fit pegs or plugs, wires, clips, clamps, or other structures for fastening two parts together.

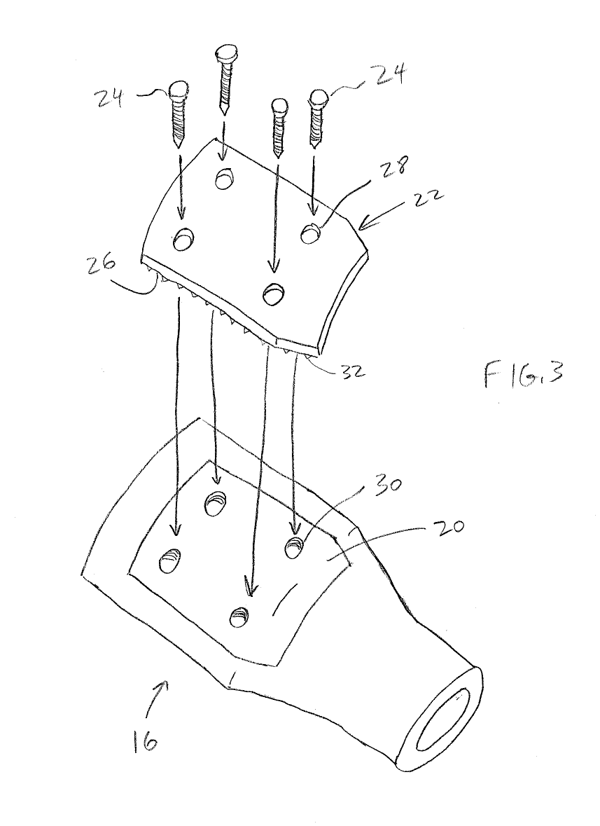

In addition, the receptor surface 20 and the attachment plate 22 are shaped and sized to provide good surface areas for contacting and securing the soft tissue 18. For example, the receptor surface 20 can have a generally rectangular shape, as shown in FIGS. 2 and 3, with a length of about ¾ inch to about 1½ inches with and a width of about ⅜ inch to about 1 inch. And the attachment plate 22 can be shaped and sized so that the attachment surface 26 completely covers the receptor surface 20 to maximize gripping contact with the soft tissue 18. Thus, the receptor surface 20 and the attachment surface 26 can have conforming shapes and/or sizes. For example, the attachment plate 22 can have a generally rectangular shape, as shown in FIG. 3, with a length of about ¾ inch to about 1½ inches and with a width of about 1 inch to about 2 inches. In alternative embodiments, the receptor surface and the attachment plate have a larger or smaller conforming size, another conforming shape, non-conforming sizes, and/or non-conforming shapes. For example, the receptor surface and/or the attachment plate can have a polygonal, oval, or other regular or irregular shape, though shapes that are elongated (relative to the implant's longitudinal axis) tend to provide good contact surface areas. In addition, the receptor and attachment surfaces 20 and 26 can be curved (about the implant's longitudinal axis) or flat, as may be desired for a given application.

Furthermore, the receptor surface 20 and/or the attachment surface 26 can have structures for gripping the soft tissue 18 to assist in securing it in place. In the depicted embodiment, for example, the attachment plate 22 has rows of teeth 32 extending from the attachment surface 26. More or fewer of the teeth 32 can be provided, as may be desired. In alternative embodiments, the receptor surface 20 and/or the attachment surface 26 have tissue-gripping structures provided by a coarse texture that provides a good gripping action on the soft tissue.

Advantageously, the attachment device may improve the stability, function, range of motion, and survival of an orthopedic prosthetic implant that is implanted during joint replacement surgery. The attachment device can be used during a primary arthroplasty, a revision procedure, or a surgery done for oncology purposes where bone resection and endoprosthetic reconstruction are required.

FIG. 6 shows an orthopedic prosthetic implant 110 with an attachment device 112 according to a second example embodiment of the present invention. In this embodiment, the implant 110 is a knee-joint arthroplasty implant used to reconstruct a hip and includes a proximal tibia component 116, as in the first example embodiment. The attachment device 112 includes a receptor surface 120, an attachment plate 122, and fasteners 124, which are similar to those of the first example embodiment. However, the attachment device 112 of this embodiment includes several modifications. The attachment plate 122 is slightly trapezoidal (though still generally rectangular), has a modified tissue-gripping tooth arrangement, and has a countersunk fastener-screw arrangement. In particular, the attachment plate 122 has a single row of tissue-gripping teeth 132 extending from its distal endwall 134. And the attachment plate 122 has countersunk recesses 136 that receive the fastener-screw heads 138 in a low-profile arrangement. In addition, the attachment device 110 and the proximal tibia component 116 are configured with the attachment holes 128 and the receptor holes 130 positioned outside the peripheral boundaries of the attachment surface and the receptor surface 120 so that the fasteners 124 do not extend through the soft tissue. Furthermore, the proximal tibia component 116 includes recesses 140 on opposite sides of the receptor surface 120, with lateral holes 142 through opposite sidewalls 144 defining the recesses. The recesses 140 and the lateral holes 142 allow for supplemental suture attachment of the tendon. Stated another way, the holes 142 are suture holes, and the recesses 140 provide access to those holes.

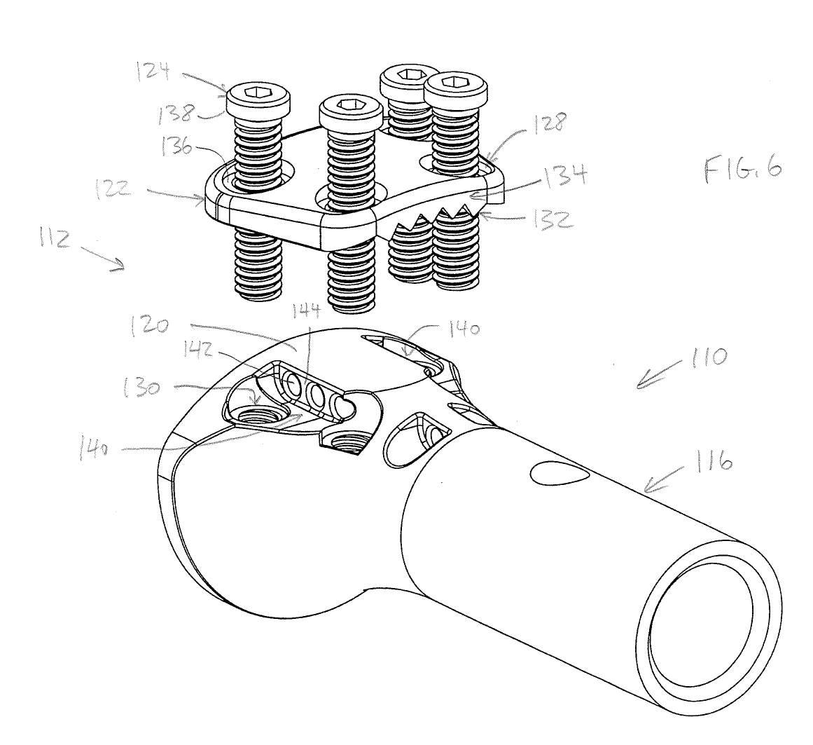

FIGS. 7 and 8 show an orthopedic prosthetic implant 210 with an attachment device 212 according to a third example embodiment of the present invention. In this embodiment, the implant 210 is a hip-joint arthroplasty implant used to reconstruct a hip. The implant 210 includes an acetabular component (e.g., a cup or shell, not shown) and a proximal femur component 216. The soft tissue 218 reattached is the vastus lateralis/gluteus medius complex, with or without a portion of the trochanter bony structure. The attachment device 212 includes a receptor surface 220, an attachment plate 222, and fasteners 224, which are similar to those of the first example embodiment. In this embodiment, however, the implant 210 and the attachment device 212 are adapted for use in this hip-replacement application. For example, the receptor surface 220 and the attachment plate 222 can have an outwardly curved proximal end, as shown. In addition, the fasteners 224 of the depicted embodiment do not extend all the way through the proximal femur component 216.

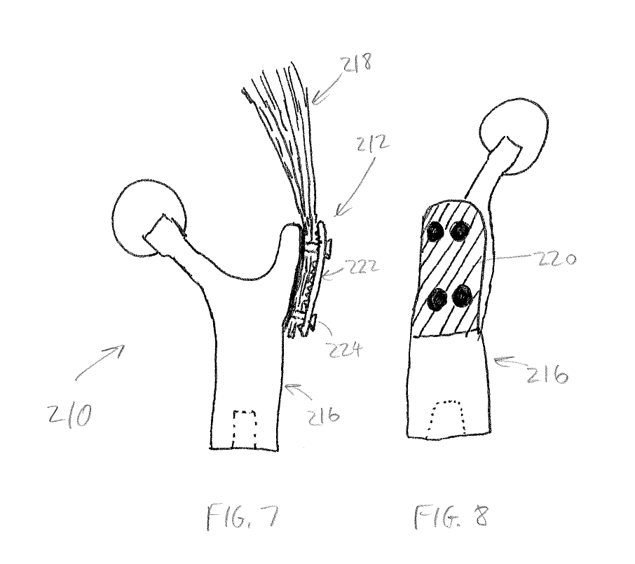

FIGS. 9 and 10 show an orthopedic prosthetic implant 310 with an attachment device 312 according to a fourth example embodiment of the present invention. In this embodiment, the implant 310 is a shoulder-joint arthroplasty implant used to reconstruct a shoulder. The implant 310 can include a scapula component 314 and a proximal humerus component 316. The soft tissue 318 reattached is the deltoid or rotator cuff complex or shoulder capsule, with or without a portion of the greater or lesser tuberosities bony structure. The attachment device 312 includes a receptor surface 320, an attachment plate 322, and fasteners 324, which are similar to those of the above-described example embodiments. In this embodiment, however, the implant 310 and the attachment device 312 are adapted for use in this shoulder-replacement application.

In alternative embodiments, the attachment device is adapted for use with other orthopedic prosthetic implants. For example, it can be adapted for use with other orthopedic prosthetic joint implants or for non-joint orthopedic prosthetic implants. For joint implants, the attachment device can be provided on both major components of the implant to attach both ends of the soft tissue, as may be desired in some applications. In some embodiments, the combination of the orthopedic implant and the attachment device are provided together as a unit. In other embodiments, the sub-combination of the attachment device or of only the attachment plate and the fasteners are provided separately for attaching soft tissue to a conventional orthopedic implant or to a bone.

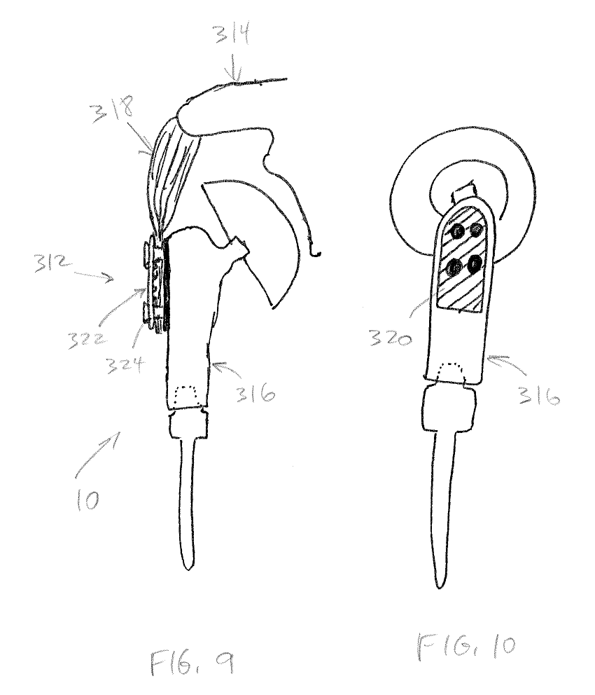

FIG. 11 shows an attachment device 412 according to a fifth example embodiment of the present invention. In this embodiment, the attachment device 412 is designed for use in attaching soft tissue to a bone 416 (as depicted) or to a conventional orthopedic prosthetic implant (not shown) without pre-formed receptor holes or a surface that could be suitably used as the receptor surface. The attachment device 412 includes an attachment plate 422 and fasteners (not shown), which are similar to those of the first example embodiment. In this embodiment, however, the attachment device 412 also includes a receptor plate 446 with a receptor surface 420. To install the attachment device 412, base holes (not shown) are formed (e.g., by drilling) in the bone or implant using the receptor holes 430 of the receptor plate 446, the attachment holes 428 of the attachment plate 422, or a template to properly position the base holes. Alternatively, where the bone or conventional implant has a surface that is suitable for use as the receptor surface, the receptor plate is not needed.

FIGS. 12-21 show modifications to the receptor surface, the attachment plate, and the fasteners according to alternative embodiments of the invention. The modifications of these alternative embodiments, as well as those of the other herein-described alternative embodiments, can be implemented in any of the above-described example embodiments.

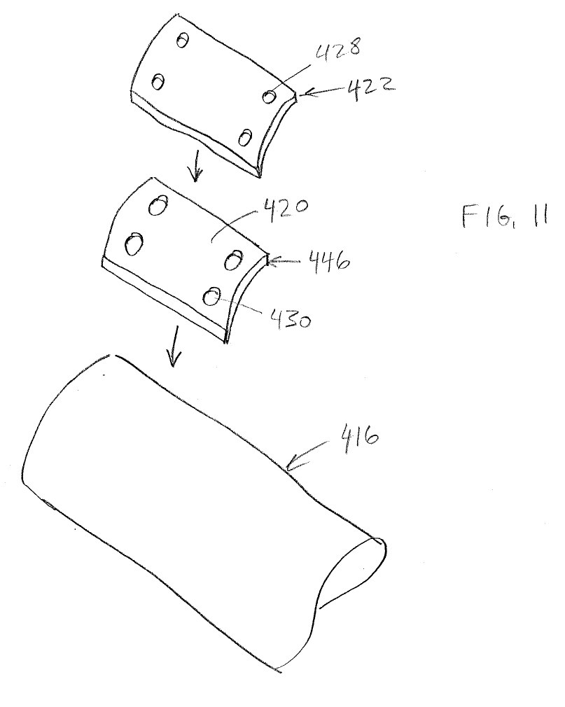

FIGS. 12 and 13 show a modified receptor surface 520 of a soft tissue attachment device 512 according to a first alternative embodiment of the invention. In this embodiment, the implant 510 and the attachment device 512 are adapted to provide a more low-profile arrangement. In particular, the receptor surface 520 is formed by a bottom-wall surface (or a portion thereof) of a recess 548 formed in the implant 510. The recess 548 can have a length (along the implant's longitudinal axis) that is greater than the length of the attachment plate 522 to provide a proximal space 550a where the soft tissue 518 enters the recess and/or a distal space 550b for excess soft tissue. Also, the recess 520 can have a depth that is greater than the thickness of the attachment plate 522 and that is selected to allow for the thickness of the soft tissue 518 while still providing a low-profile arrangement with the outer surface of the attachment plate not extending beyond the outer surface of the implant 510. In addition, the fasteners 524 can be countersunk into the attachment plate, as shown, to contribute to the low profile.

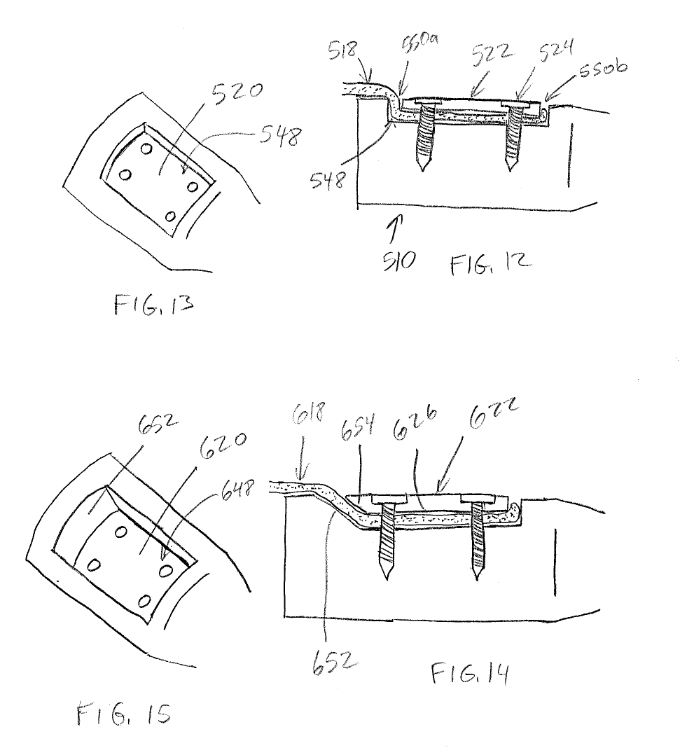

FIGS. 14 and 15 show a modified receptor surface 620 and attachment plate 622 of a soft tissue attachment device 612 according to a second alternative embodiment of the invention. This attachment device 612 is similar to that of the first alternative embodiment, except that the recess 648 has a ramped proximal end 652 and the attachment surface 626 of the attachment plate 622 has a ramped proximal end 654. The receptor and attachment ramps 652 and 654 are ramped downwardly in the proximal-to-distal direction. The receptor and attachment ramps 652 and 654 can be curved, linear, or have another regular or irregular shape. While receptor and attachment ramps 652 and 654 with conforming surfaces works well, they can be provided with different non-conforming surfaces such as by including soft-tissue gripping structures. This ramped arrangement can reduce or eliminate bending stresses on and pinching of the soft tissue.

FIG. 16 shows a modified receptor surface 720 of a soft tissue attachment device 712 according to a third alternative embodiment of the invention. This attachment device 712 is similar to that of the first example embodiment, except that the soft-tissue gripping teeth 732 of the attachment plate 722 are angled distally. This arrangement provides an excellent gripping action on the soft tissue.

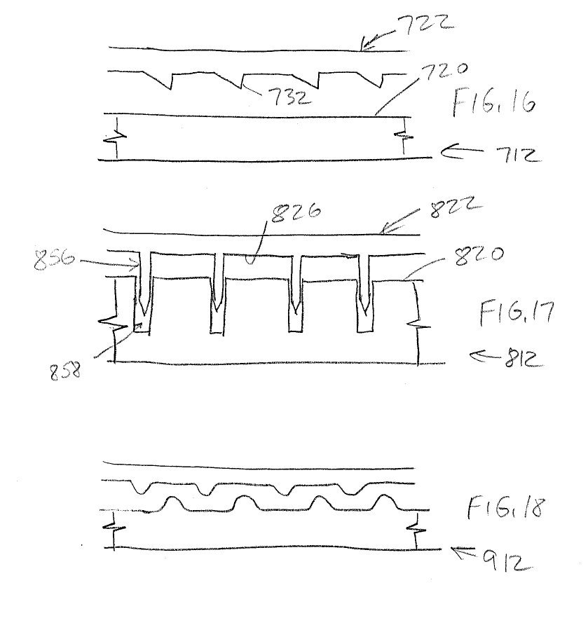

FIG. 17 shows a modified receptor surface 820 and attachment plate 822 of a soft tissue attachment device 812 according to a fourth alternative embodiment of the invention. This attachment device 812 is similar to those of the other embodiments described herein, except that it has a different soft-tissue gripping arrangement. The attachment device 812 has a plurality of spikes 856 extending from the attachment surface 826 of the attachment plate 822 and a plurality of spike holes 858 formed in the receptor surface 820 in aligned positions to receive at least a portion of the length of the spikes. When the attachment plate 822 is installed, the spikes 856 puncture and push all the way through the soft tissue until they are received in the spike holes 858. This arrangement provides for excellent securement of the soft tissue. In other alternative embodiments, the spikes extend from the receptor surface and the spike holes are formed in the attachment surface, or the attachment and receptor surfaces both have spikes and both have cooperating spike holes for receiving the other plate's spikes. In yet other alternative embodiments, the spikes do not have sharp points and/or they do not extend through the soft tissue.

FIG. 18 shows a modified receptor surface 920 and attachment plate 922 of a soft tissue attachment device 912 according to a fifth alternative embodiment of the invention. This attachment device 912 is similar to those of the other embodiments described herein, except that it has a different soft-tissue gripping arrangement. The attachment device 912 has a plurality of protruding members 960 extending inwardly from the attachment surface 926 of the attachment plate 922 and a plurality of protruding members 962 extending outwardly from the receptor surface 920, with the protruding members not in alignment with each other. Thus, the protruding members of one surface are interposed between the protruding members of the other surface. The protruding members 960 and 962 can be provided by pegs, ridges, teeth, spikes, or other protruding structural members that will provide a good gripping action on soft tissue when compressed against it. This arrangement also provides for excellent securement of the soft tissue. In other alternative embodiments, the protruding members extend from only the attachment surface or the receptor surface, and the other plate has cooperating recesses that receive them.

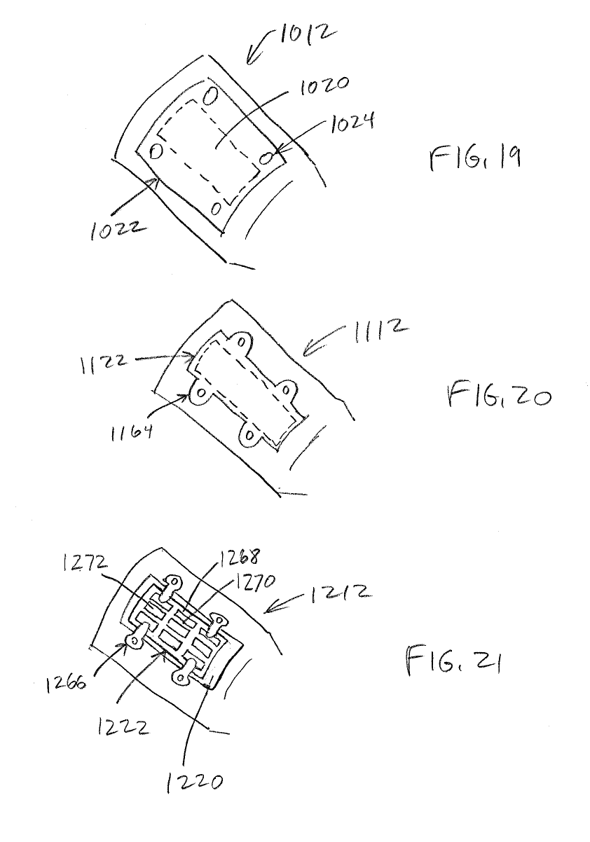

FIG. 19 shows a modified attachment plate 1022 of a soft tissue attachment device 1012 according to a sixth alternative embodiment of the invention. This attachment device 1012 has a receptor surface 1020 and fasteners 1024 that are similar to those of the other embodiments described herein. In this embodiment, however, the receptor surface 1020 and the attachment plate 1022 are configured with the receptor and attachment holes outside of the peripheral boundary of the receptor surface so that the fasteners 1024 do not extend through the soft tissue.

FIG. 20 shows a modified attachment plate 1122 of a soft tissue attachment device 1112 according to a seventh alternative embodiment of the invention. This attachment device 1112 is similar to that of the sixth alternative embodiment, except that the attachment plate 1122 is smaller and has tabs 1164 extending outwardly from it, with the attachment holes formed in the tabs.

FIG. 21 shows a modified soft tissue attachment device 1212 according to an eighth alternative embodiment of the invention. This attachment device 1212 has a receptor surface 1220, an attachment plate 1222, and fasteners 1224 that are similar to those of the other embodiments described herein. In this embodiment, however, the fasteners 1224 include tabs (e.g., hooks or clamps) with the attachment holes formed in them and with extensions that overlap the attachment plate 1222 to hold it in place. Also, the attachment plate 1222 is provided by a grid-like lattice structure, with a peripheral frame member 1268 and interior cross-members 1270 forming interior spaces 1272 that the soft tissue can occupy.

It is to be understood that this invention is not limited to the specific devices, methods, conditions, or parameters of the example embodiments described and/or shown herein, and that the terminology used herein is for the purpose of describing particular embodiments by way of example only. Thus, the terminology is intended to be broadly construed and is not intended to be unnecessarily limiting of the claimed invention. For example, as used in the specification including the appended claims, the singular forms “a,” “an,” and “the” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

While the claimed invention has been shown and described in example forms, it will be apparent to those skilled in the art that many modifications, additions, and deletions can be made therein without departing from the spirit and scope of the invention as defined by the following claims.

What is claimed is:

1. An orthopedic prosthetic implant to which soft tissue is attached, comprising:
an implant component defining a receptor surface that accepts thereon a portion of the soft tissue;
an attachment plate defining an attachment surface that at least partially covers the soft tissue portion; and
at least one fastener that secures the attachment plate to the implant component with the soft tissue portion compressed therebetween.

2. The implant of claim 1, further comprising tissue-gripping structures that are formed on the attachment surface, the receptor surface, or both, and that grip and hold in place the soft tissue portion.

3. The implant of claim 2, wherein the tissue-gripping structures include teeth.

4. The implant of claim 2, wherein the tissue-gripping structures include spikes extending from the attachment surface or the receptor surface, and spike-holes formed in the other of the attachment surface or the receptor surface and positioned in alignment with the spikes so that, with the attachment plate secured to the implant component, the spikes extend into the spike-holes.

5. The implant of claim 1, wherein the attachment surface is the same size or larger than the receptor surface.

6. The implant of claim 1, wherein the receptor surface is formed within a recess defined in the implant component and into which the soft tissue portion and at least a portion of the attachment plate are received.

7. The implant of claim 6, wherein the receptor surface includes a ramped entry end where the soft tissue portion enters the recess.

6. The implant of claim 7, wherein the attachment surface includes a ramped entry end that generally conforms to the receptor surface ramped entry end.

9. The implant of claim 1, wherein the receptor surface and the attachment surface are generally rectangular.

10. The implant of claim 1, wherein the at least one fastener is provided by two or more fasteners, the attachment plate defines two or more attachment holes, and the implant component defines two or more receptor holes, wherein, with the attachment plate secured to the implant component, the attachment holes and the receptor holes are aligned with the fasteners extending into the aligned attachment and receptor holes.

11. The implant of claim 10, wherein the attachment holes are formed in the attachment surface and the receptor holes are formed in the receptor surface so that, with the attachment plate secured to the implant component, the fasteners extend through the soft tissue portion.

12. A method attaching soft tissue to the orthopedic prosthetic implant of claim 1, comprising:
positioning the soft tissue portion on the receptor surface;
positioning the attachment plate to at least partially cover the soft tissue position; and
securing the attachment plate to the implant component with the at least one fastener.

13. An orthopedic prosthetic implant to which soft tissue is attached, comprising:
an implant component defining a generally rectangular receptor surface that accepts thereon a portion of the soft tissue;
an attachment plate defining a generally rectangular attachment surface that covers the soft tissue portion, wherein the attachment surface is the same size or larger than the receptor surface;
tissue-gripping structures formed on the attachment surface, the receptor surface, or both, that grip and hold in place the soft tissue portion; and
two or more fasteners that secure the attachment plate to the implant component with the soft tissue portion compressed therebetween.

14. The implant of claim 12, wherein the tissue-gripping structures include spikes extending from the attachment surface or the receptor surface, and spike-holes formed in the other of the attachment surface or the receptor surface and positioned in alignment with the spikes so that, with the attachment plate secured to the implant component, the spikes extend into the spike-holes.

15. The implant of claim 12, wherein the receptor surface is formed within a recess defined in the implant component and into which the soft tissue portion and at least a portion of the attachment plate are received.

16. The implant of claim 13, wherein the receptor surface includes a ramped entry end where the soft tissue portion enters the recess and the attachment surface includes a ramped entry end that generally conforms to the receptor surface ramped entry end.

17. The implant of claim 12, wherein the attachment plate defines two or more attachment holes in the attachment surface and the implant component defines two or more receptor holes in the receptor surface, wherein, with the attachment plate secured to the implant component, the attachment holes and the receptor holes are aligned with the fasteners extending through the soft tissue portion and into the aligned attachment and receptor holes.

18. A device for attaching soft tissue to an orthopedic prosthetic implant or to a bone, comprising:
an attachment plate defining an attachment surface that at least partially covers at least a portion of the soft tissue; and
at least one fastener that secures the attachment plate to the implant or the bone with the soft tissue portion compressed therebetween.

19. The implant of claim 18, further comprising a receptor plate defining a receptor surface that accepts thereon the soft tissue, wherein the attachment surface at least partially covers the soft tissue portion and the receptor surface.

20. A method attaching the soft tissue portion of claim 19 to the implant or bone of claim 19 using the device of claim 19, comprising:
positioning the soft tissue portion on the receptor surface;
positioning the attachment plate to at least partially cover the soft tissue position; and
securing the attachment plate to the implant or bone with the at least one fastener.

21. The implant of claim 18, wherein the implant or the bone defines a receptor surface that accepts thereon the soft tissue, wherein the attachment surface at least partially covers the soft tissue portion and the receptor surface.

22. A method attaching the soft tissue portion of claim 19 to the implant or bone of claim 19 using the device of claim 19, comprising:
positioning the soft tissue portion on the receptor surface;
positioning the attachment plate to at least partially cover the soft tissue position; and
securing the attachment plate to the implant or bone with the at least one fastener.

23. The implant of claim 18, further comprising tissue-gripping structures that are formed on the attachment surface and that grip and hold in place the soft tissue portion, wherein the attachment surface includes a ramped entry end under which the soft tissue portion is positioned, wherein the attachment surface is generally rectangular, wherein the attachment plate defines two or more attachment holes in the attachment surface, and wherein, with the attachment plate secured to the implant or the bone, the fasteners extend through the soft tissue portion and into the attachment holes.
